(19) United States
(12) Patent Application Publication
(10) Pub. No.: US 2008/0044927 A1
(43) Pub. Date: Feb. 21, 2008

(54) MEDICAL TEST STRIP

(76) Inventor: Ching Ho Lien, Taipei City (TW)

Correspondence Address: Cheng Ho Lien, 235 Chung - Ho Box 8-24 Taipei, Taipei, 235, TW

(21) Appl. No.: 11/844,361

(22) Filed: Aug. 24, 2007

Related U.S. Application Data
(63) Continuation-in-part of application No. 10/285,057, filed on Oct. 30, 2002, now abandoned.

(51) Int. Cl. G01N 33/558

(52) U.S. Cl. 436/514

(57) ABSTRACT

A test strip comprises a substrate having a long and narrow rectangular shape and the substrate having no hole thereon; a first sample-collection pad adhered to one side of the substrate, the first sample-collection pad having a side notch; the notch and the substrate being formed with a blood sample receiving chamber; the first sample-collection pad having a rectangular shape; and the side notch being transverse to a side of the substrate; the side notch having an opening at one side of the substrate; a size of the first sample-collection pad being smaller than that of the substrate; and at least one second sample-collection pad adhered to an outer surface of the first sample-collection pad in parallel to the substrate and defining a blood sample entrance accessible to the blood sample receiving chamber; and the at least one second sample-collection pad having a rectangular shape.

The present invention is a continuation in part of U.S. patent application Ser. No. 10/285,057 which is assigned to and invented by the inventor and applicant of the present invention. Thus, the content of U.S. patent application Ser. No. 10/285,057 is incorporated into the present invention as a part of the present invention.

### BACKGROUND OF THE INVENTION

1. Field of the Invention

The present invention relates to test strips for medical examination and, more particularly, to such a test strip, which can absorb blood sample from different directions.

2. Description of the Related Art

Various portable medical examination apparatus are commercially available for examining a particular item or multiple items of blood substances with test strips. With respect to blood sample collection, regular test strips for use with medical examination apparatus include two types, i.e., the direct blood collection type and the indirect blood collection type. A test strip of direct blood collection type must be attached to the punched point of the patient's finger to collect the blood. When a test strip of indirect blood collection type is used, the user or examiner drops blood sample to the test strip with an implement. Because the amount of blood sample applied to the test strip affect the examination result. The examiner must carefully drop the blood sample to the test strip.

### SUMMARY OF THE INVENTION

The present invention has been accomplished under the circumstances in view. It is the main object of the present invention to provide a test strip, which can collect blood sample from the top as well as from the side.

According to one embodiment of the present invention, a test strip is provided, which comprises a substrate having a long and narrow rectangular shape and the substrate having no hole thereon; a first sample-collection pad adhered to one side of the substrate, the first sample-collection pad having a side notch; the notch and the substrate being formed with a blood sample receiving chamber; the first sample-collection pad having a rectangular shape; and the side notch being transverse to a side of the substrate; the side notch having an opening at one side of the substrate; a size of the first sample-collection pad being smaller than that of the substrate; and at least one second sample-collection pad adhered to an outer surface of the first sample-collection pad in parallel to the substrate and defining a blood sample entrance accessible to the blood sample receiving chamber; and the at least one second sample-collection pad having a rectangular shape.

Furthermore the present invention provides a test strip comprising: a substrate having a long and narrow rectangular shape and the substrate having no hole thereon; a first sample-collection pad adhered to one side of the substrate, the first sample-collection pad; the first sample-collection pad having a rectangular shape; a size of the first sample-collection pad being smaller than that of the substrate; and at least one second sample-collection pad adhered to an outer surface of the first sample-collection pad in parallel to the substrate and defining a blood sample entrance accessible to the blood sample receiving chamber; the second sample-collection pad having a rectangular shape and a size of the second sample-collection pad being greater than that of the second sample-collection pad.

Moreover, the second-sample collection pad has three notches in three of the four sides thereof; each notch has a semi-round shape with an opening at a respective side of the pad; when the test strip assembled, the notches are disposed in communication with the blood sample receiving chamber defined by the first and second sample-collection pads and the substrate.

### BRIEF DESCRIPTION OF THE DRAWINGS

FIG. 1 is an exploded view of a test strip according to the first embodiment of the present invention.

FIG. 2 is an elevational assembly view of the test strip according to the first embodiment of the present invention.

FIG. 3 is a side view of the test strip according to the first embodiment of the present invention.

FIG. 4 is a top view of the test strip according to the first embodiment of the present invention.

FIG. 5 is an exploded view of a test strip according to the second embodiment of the present invention.

FIG. 6 is an elevational assembly view of the test strip according to the second embodiment of the present invention.

FIG. 7 is an exploded view of a test strip according to the third embodiment of the present invention.

FIG. 8 is an elevational assembly view of the test strip according to the third embodiment of the present invention.

FIG. 9 is an exploded view of a test strip according to the fourth embodiment of the present invention.

FIG. 10 is an elevational assembly view of the test strip according to the fourth embodiment of the present invention.

FIG. 11 is an exploded view of a test strip according to the fifth embodiment of the present invention.

FIG. 12 is an elevational assembly view of the test strip according to the fifth embodiment of the present invention.

### DETAILED DESCRIPTION OF THE PREFERRED EMBODIMENT

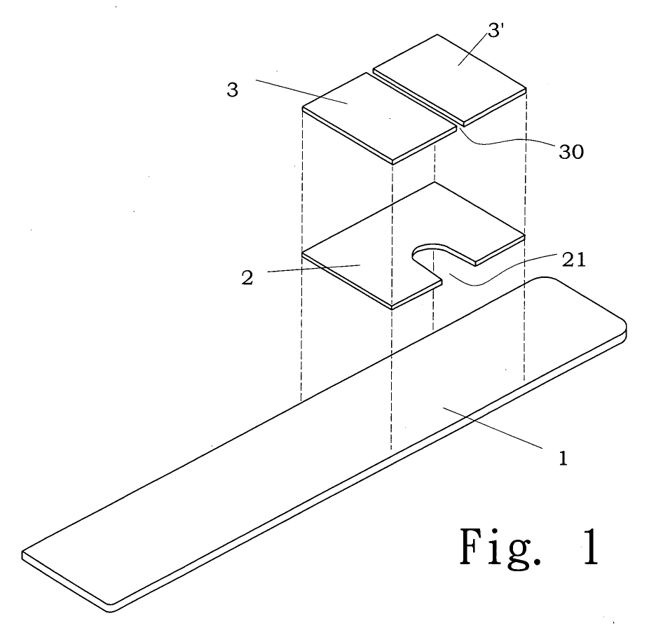

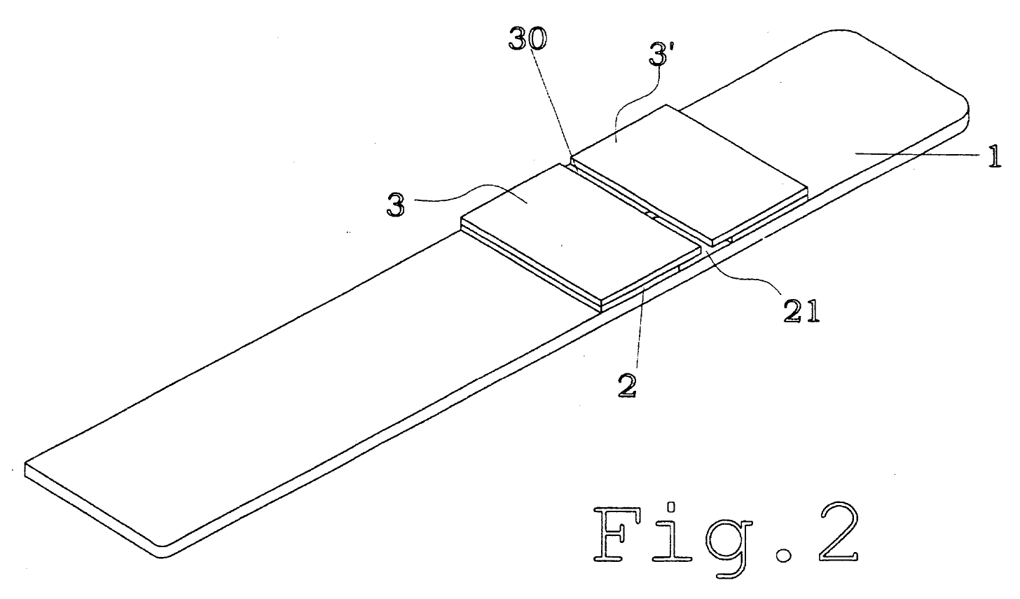

Referring to FIGS. 1 and 2, a test strip is shown. The test strip comprises the following elements. A substrate 1 has an upper major surface and has a narrow elongated rectangular shape without any hole therein. A first sample-collection pad 2 is adhered to the upper major surface of the substrate 1 and has a rectangular shape with a side notch 21. The side notch 21 is transverse to a side of the substrate. The notch 21 has an opening oriented toward the side of the substrate. A size of the first sample-collection pad 2 is smaller than that of the substrate 1. Two second sample-collection pads 3, 3′ are adhered to an upper surface of the first sample-collection pad 2 in parallel to the substrate. Each of the second sample-collection pads 3 has a rectangular shape. A blood sample receiving chamber is bounded by the substrate 1, the second sample-collection pads 3, and the side notch 21. A side blood sample entrance defined by the substrate, the second sample-collection pad, and the opening of the side notch is fluidically communicated with the blood sample receiving chamber for receiving blood by direct contact with the blood sample receiving chamber for receiving blood by direct contact with a blood source. A gap 30 is formed between the two second sample-collection pads 3 as a top blood sample entrance between the two second sample collection pads 3. In use, the gap 30 is fluidically communicated with a blood sample receiving chamber for receiving blood dropped onto the upper surface of the second sample-collection pads 3, 3′.

In this embodiment, preferably, the side notch 21 has a smoothly arched profile. In assembly, the first sample-collection pad 2 adhered to the substrate 1, and the side notch 21 with the substrate 1 is formed with a blood sample receiving chamber. Then the second sample-collection pads 3 and 3′ is adhered to the first sample-collection pad 2. The narrow gap 30 is left between the second sample-collection pads 3 and 3′ in communication with the side notch 21 of the first sample-collection pad 2.

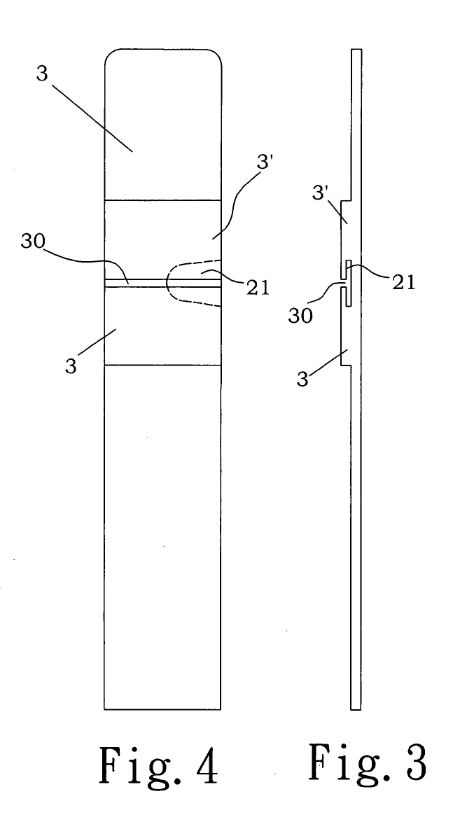

Referring to FIGS. 3 and 4, when the test strip inserted into the test apparatus and the reagent permeated into the sample-collection pads 2, 3, and 3′, the test strip is attached to the punched point of the patient's finger with the side notch 21 of the first sample-collection pad 2 approached the blood flowing out of the punched point of the patient finger, enabling the sample blood to be guided into the receiving chamber defined by the substrate 1 and the first sample-collection pad 2 and then absorbed by the sample-collection pads 2, 3, and 3′ to make a chemical reaction with the reagent in the sample-collection pads 2, 3, and 3′, for enabling the test apparatus to read the value of the assigned substance in the test sample (blood). In another test method, the user or examiner can drop the test sample (patient's blood or serum) into the gap 30 between the second sample-collection pads 3 and 3′, enabling the test sample to be absorbed by the sample-collection pads 2, 3, and 3′ for further examination.

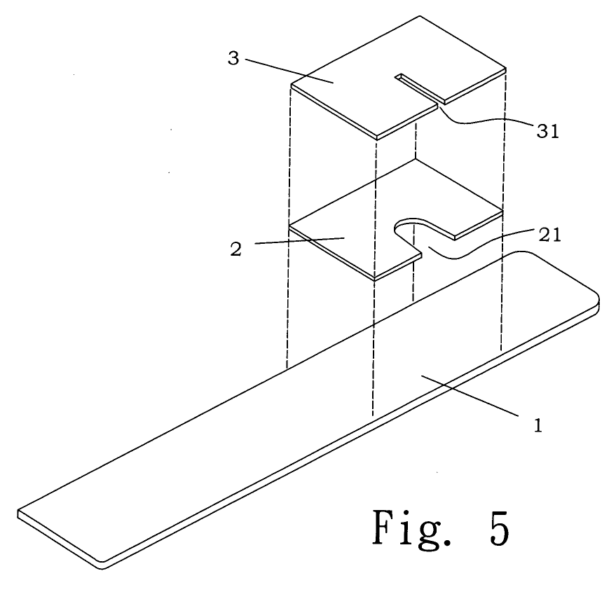

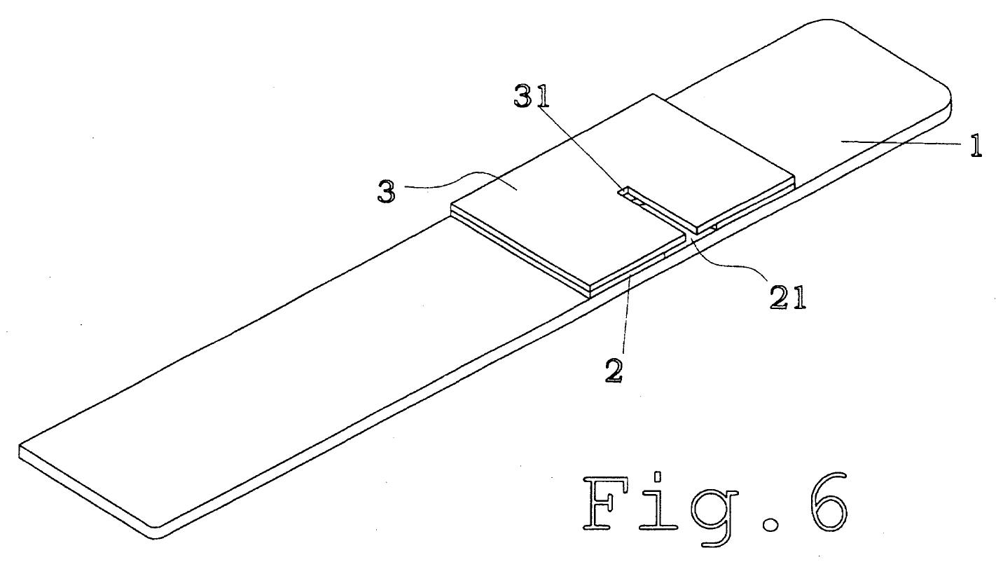

FIGS. 5 and 6 show a test strip according to the second embodiment of the present invention. According to this embodiment, a test strip comprises the following elements.

A substrate 1 has an upper major surface and has a rectangular shape without any hole therein. A first sample-collection pad 2 is adhered to the upper major surface of the substrate 1 and has a rectangular shape with a side notch 21. The side notch 21 is transverse to a side of the substrate. The notch 21 has an opening oriented toward the side of the substrate. A size of the first sample-collection pad 2 is smaller than that of the substrate 1. A second sample-collection pad 3 is adhered to an upper surface of the first sample-collection pad 2 in parallel to the substrate. The second sample-collection pad 3 has a rectangular shape. The second sample-collection pad 3 and the first sample-collection pad 2 have the same size. The second sample-collection pad 3 has a narrow transverse slot 31 extended to one side in communication with the side notch 21. This embodiment achieves the same effect as the aforesaid first embodiment. Same as the test strip of the aforesaid first embodiment, the test strip of the second embodiment can be inserted into a test apparatus to absorb the reagent and then to collect the blood sample from the patient. Alternatively, the test strip can absorb the test sample (the patient's blood) at first and then inserted into the examination apparatus to examine the value of the assigned substance in the patient blood.

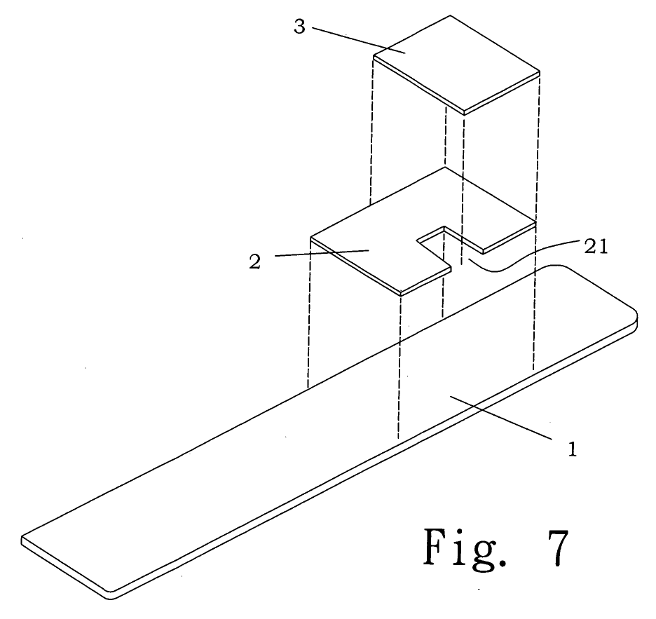

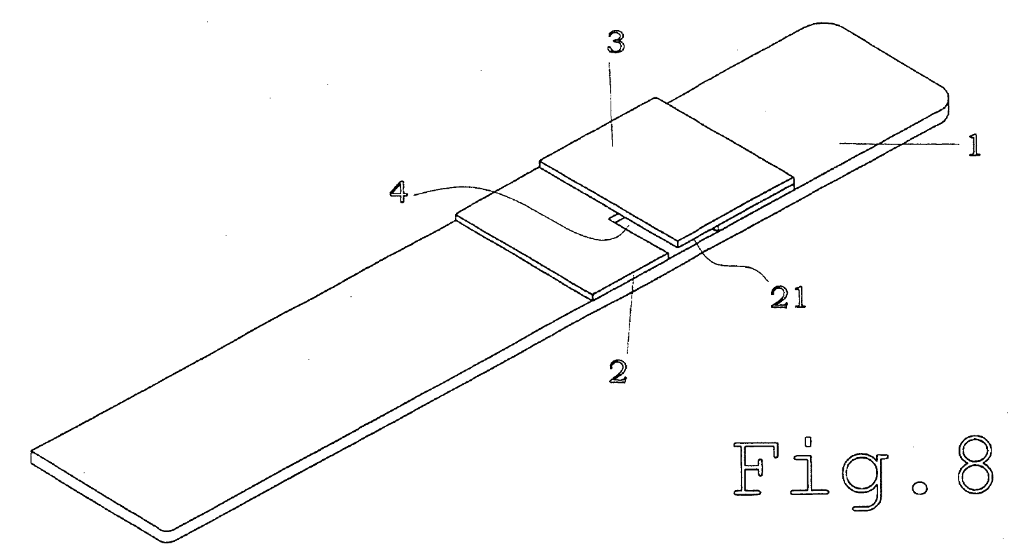

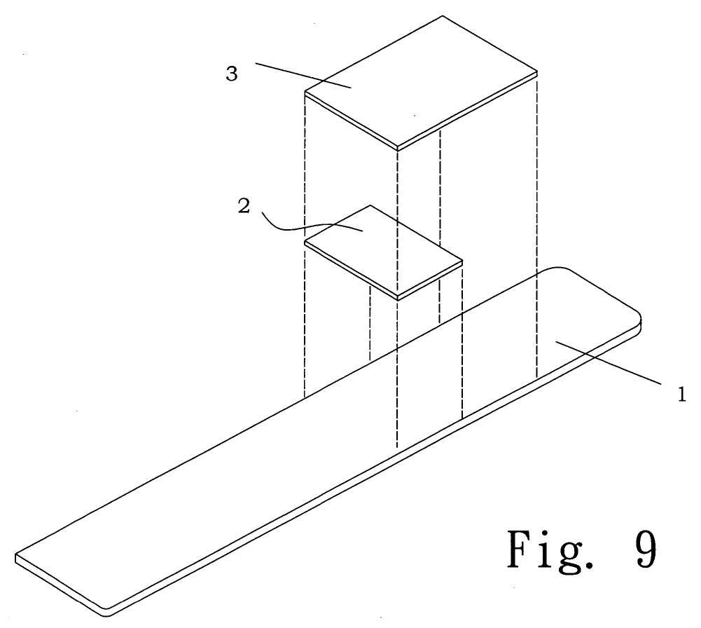

FIGS. 7 and 8 show a test strip according to the third embodiment of the present invention. According to this embodiment, the test strip comprises a substrate 1, a first sample-collection pad 2 adhered to one side of the substrate 1, and a second sample-collection pad 3 adhered to the outer surface of the first sample-collection pad 2. The substrate 1 has a long narrow rectangular shape. The first sample-collection pad 2 has a rectangular shape with a side notch 21 opened at one long side. The side notch 21 of the first sample-collection pad 2 according to this embodiment has a rectangular shape. The second sample-collection pad 3 has a rectangular shape and is relatively smaller than the first sample-collection pad 2. The second sample-collection pad 2 is adhered to a part of the outer surface of the second sample-collection pad 2. In assembly, the second sample-collection pad 3 adhered to the first sample-collection pad 1. The second sample-collection pad 3 and the first sample-collection pad 2 define an entrance 4 accessible to the blood sample receiving chamber (the side notch 21 of the first sample-collection pad 2). This embodiment can be used to examine a particular item of the patient blood in same manner as the aforesaid first and second embodiments.

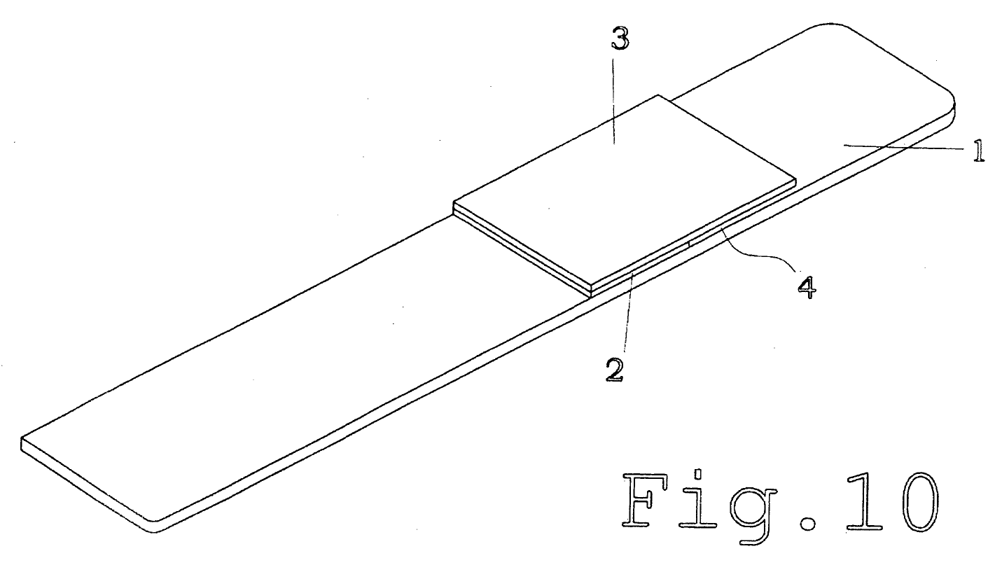

FIGS. 9 and 10 show a test strip according to the fourth embodiment of the present invention. According to this embodiment, the test strip comprises a substrate 1, a first sample-collection pad 2 adhered to one side of the substrate 1, and a second sample-collection pad 3 adhered to the first sample-collection pad 2. The second sample-collection pad 3 is relatively greater than the first sample-collection pad 2. When assembled, the second sample-collection pad 3 and the first sample-collection pad 2 define with the substrate 1 a blood sample receiving chamber having an entrance 4 in three sides.

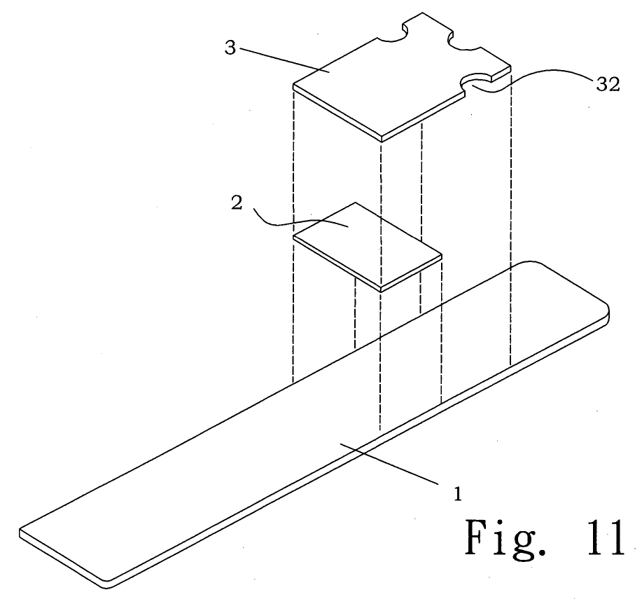

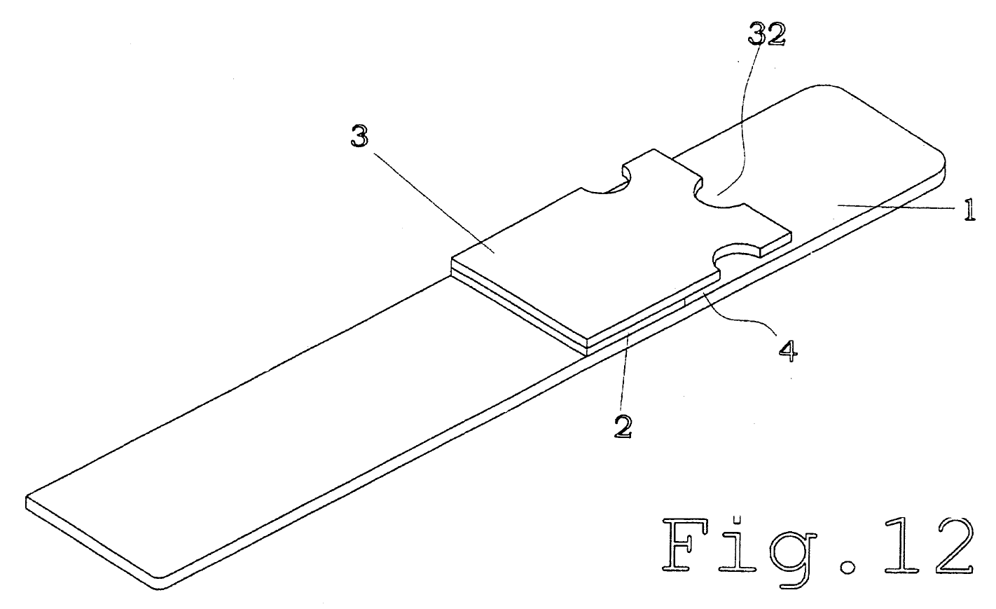

FIGS. 11 and 12 show a test strip according to the fifth embodiment of the present invention. This embodiment is similar to the aforesaid fourth embodiment with the exception of the shape of the second sample-collection pad 3. The substrate 1 has a long narrow rectangular shape. The first sample collection pad 2 and the second sample collection pad 3 have oblong rectangular shapes. The second-sample collection pad 3 has three notches 32 in three of the four sides thereof. Each notch has a semi-round shape with an opening at a respective side of the pad 3. When the test strip assembled, the notches 32 are disposed in communication with the blood sample receiving chamber defined by the sample-collection pads 2 and 3 and the substrate 1.

A prototype of test strip has been constructed with the features of FIGS. 1 about 12. The test strip functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A test strip comprising:
a substrate having a long and narrow rectangular shape and the substrate having no hole thereon;
a first sample-collection pad adhered to one side of the substrate, the first sample-collection pad having a side notch; the notch and the substrate being formed with a blood sample receiving chamber; the first sample-collection pad having a rectangular shape; and the side notch being transverse to a side of the substrate; the side notch having an opening at one side of the substrate; a size of the first sample-collection pad being smaller than that of the substrate; and
at least one second sample-collection pad adhered to an outer surface of the first sample-collection pad in parallel to the substrate and defining a blood sample entrance accessible to the blood sample receiving chamber; and the at least one second sample-collection pad having a rectangular shape.

2. The test strip as claimed in claim 1, wherein the side notch has a semi-round edge at a bottom side of the notch; and there are two second sample-collection pads which are arranged in parallel parts and are adhered to the first sample-collection pad; a narrow gap is formed between the two second sample-collection pads so as to form the blood sample entrance.

3. The test strip as claimed in claim 1, wherein the second sample-collection pad has an elongated slot extended to one side thereof and forming the blood sample entrance as the second sample-collection pad adhered to the first sample-collection pad.

4. The test strip as claimed in claim 1, wherein the side notch has a rectangular shape; and the second sample-collection pad covers a upon the first sample-collection pad and only a half part of the notch is covered by the second sample-collection pad.

5. A test strip comprising:
a substrate having a long and narrow rectangular shape and the substrate having no hole thereon;
a first sample-collection pad adhered to one side of the substrate; the first sample-collection pad having a rectangular shape; a size of the first sample-collection pad being smaller than that of the substrate; and
at least one second sample-collection pad adhered to an outer surface of the first sample-collection pad in parallel to the substrate and defining a blood sample entrance accessible to the blood sample receiving chamber; the second sample-collection pad having a rectangular shape and a size of the second sample-collection pad being greater than that of the second sample-collection pad.

6. The test-strip as claimed in claim 1, wherein the second-sample collection pad has three notches in three of the four sides thereof; each notch has a semi-round shape with an opening at a respective side of the pad; when the test strip assembled, the notches are disposed in communication with the blood sample receiving chamber defined by the first and second sample-collection pads and the substrate.
